(19) United States
(12) Patent Application Publication
(10) Pub. No.: US 2016/0120557 A1
(43) Pub. Date: May 5, 2016

(54) MEDICAL DEVICE FOR REMOVING PARTICLES

(71) Applicant: Boston Scientific Scimed, Inc., Maple Grove, MN (US)

(72) Inventors: James M. GODDARD, Pepperell, MA (US); Ronald CIULLA, Westford, MA (US)

(73) Assignee: Boston Scientific Scimed, Inc., Maple Grove, MN (US)

(21) Appl. No.: 14/930,935

(22) Filed: Nov. 3, 2015

Related U.S. Application Data
(60) Provisional application No. 62/075,639, filed on Nov. 5, 2014.

(51) Int. Cl. A61B 17/22; A61M 1/00

(52) U.S. Cl. CPC A61B 17/22; A61B 19/54; A61M 1/0058; A61M 1/0062; A61B 2017/22079; A61B 2019/5425; A61B 2019/5466

(57) ABSTRACT

The present disclosure is directed to a medical device. Systems and methods are provided for removing particles, stone fragments and/or stone dust from a patient. The medical device described herein may work by positioning within the kidney, a catheter to flush stone dust. The catheter may include a dual lumen tube which provides fluid through one lumen to irrigate a target area and provides suction through a second lumen to carry out the irrigation fluid along with stone dust. This stone dust may be the result of a previously performed stone dusting. The irrigation fluid may be introduced with a pulsed flow. The pulsed flow irrigation may facilitate the removal of small stone fragments. The suction may be applied by a vacuum at to evacuate the target area at a flow rate balanced with the flow rate of the introduction of the irrigation fluid.

### CROSS-REFERENCE TO RELATED APPLICATIONS

The application claims the benefits of priority from U.S. Provisional Application No. 62/075,639, filed on Nov. 5, 2014, which is incorporated by reference herein in its entirety.

### TECHNICAL FIELD

Embodiments of the present disclosure relate generally to medical devices. More particularly, embodiments of the disclosure relate to medical devices for use in medical applications, such as, for example, removing particles, stone fragments and/or stone dust from a patient. Embodiments of the disclosure also cover methods of using such instruments.

### BACKGROUND OF THE DISCLOSURE

The incidence of hospitalization for the removal of urinary calculi, commonly referred to as kidney stones, has been estimated as 200,000 cases per year. A vast majority of these patients pass their stones spontaneously; however, in the remainder, the kidney stone(s) become impacted in the ureter, a muscle tube joining the kidney to the bladder. An impacted kidney stone is a source of intense pain and bleeding, a source of infection and, if a stone completely blocks the flow of urine for any extended length of time, can cause a loss of a kidney.

Recently, various methods have been utilized to break the stone into smaller fragments. One such method is stone dusting. Stone dusting is used by some urologist to fragment and evacuate stones from a kidney and is often performed by a laser. In some cases, the intense light energy from the laser breaks the stone into increasingly smaller pieces. Rather than breaking up the stone into chunks, which are removed by baskets, dusting generates very small fragments that are capable of being passed naturally. However, in some cases, these small stone fragments may not pass naturally. For example, the stone fragments may collect in an area of the kidney where they are less likely to flow out naturally, such as the lower pole of the kidney. In theory, any of these small stone fragments that do not evacuate through natural urine flow, could be a seed for new stone growth. The disclosure addresses the above-mentioned process and other problems in the art.

### SUMMARY OF THE DISCLOSURE

Embodiments of the present disclosure provide methods for removing particles from the human body.

Additional objects and advantages of the claimed invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

In one example, a method of removing stone dust from a patient's body may include positioning a medical device at a target area within the urinary tract, the medical device including a first lumen and a second lumen; introducing fluid through the first lumen; and applying suction to the second lumen.

Examples of the method may additionally and/or alternatively include one or more of the following features: a flow rate through the first lumen is balanced with the flow rate through the second lumen; pulsing a flow of the fluid through the first lumen; the introduction of fluid through the first lumen includes pumping the fluid from a fluid source; the flow of the fluid is pulsed in an aperiodic oscillatory pattern; the flow of the fluid is pulsed in a periodic pattern; a distal opening of the first lumen is at the same axial position as a distal opening of the second lumen; a distal opening of the first lumen is positioned proximally to a distal opening of the second lumen.

In another example, a method of using a medical device to remove a particle from a subject's body, may include the medical device including a first lumen and a second lumen; positioning the medical device proximal to a target area; pulsing fluid through the first lumen; and applying suction to the second lumen.

Examples of the method may additionally and/or alternatively include one or more of the following features: the target area is within a kidney; the target area is the lower pole of the kidney; the fluid is pulsed in an aperiodic oscillatory pattern; the fluid is pulsed in a periodic pattern; a distal opening of the first lumen is at the same axial position as the distal opening of the second lumen; a distal opening of the first lumen is positioned proximally to the distal opening of the second lumen.

In another example, a method for removing fragments of a kidney stone may include after the completion of a medical procedure that results in small fragments of at least one kidney stone, positioning a medical device at a target area within the urinary tract; introducing fluid; and applying suction.

Examples of the method may additionally and/or alternatively include one or more of the following features: the target area is the location of the completed medical procedure; a third lumen configured to receive at least one of an instrument, a medical device, and a guidewire; wherein positioning the medical device, further comprises: guiding the medical device over a guidewire; and the medical device includes radiopaque or sonoreflective markers.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 illustrates an exemplary embodiment of a medical device, including a tube, a handle portion, a fluid supply assembly, and a vacuum source;

FIG. 2 illustrates an exemplary embodiment of a cross-section of the tube taken at 2-2 of FIG. 1;

FIG. 3 illustrates an alternative exemplary embodiment of a cross-section of the tube taken at 2-2 of FIG. 1;

FIG. 4 illustrates an exemplary embodiment of the medical device extending into a patient's body;

FIG. 5 illustrates an exemplary embodiment of a distal end of the medical device of FIG. 1;

FIG. 6 illustrates an alternative exemplary embodiment of a distal end of the medical device of FIG. 1; and

FIG. 7 is a block diagram of an exemplary method of using medical devices disclosed herein.

### DESCRIPTION OF THE EMBODIMENTS

Reference will now be made in detail to exemplary embodiments of the disclosure, examples of which are illustrated in the accompanying drawings. Wherever possible, the same reference numbers will be used throughout the drawings to refer to the same or like parts.

### Overview

Embodiments of the present disclosure relate to systems and methods for removing particles from the body. The medical device described herein may work by positioning a catheter within the kidney to flush particles such as stone dust. More specifically, in exemplary embodiments, the catheter includes a dual lumen tube which provides fluid to irrigate a target area through one lumen and provides suction to carry out the irrigation fluid along with the particles through a second lumen. The irrigation fluid may be introduced with a pulsed flow. The pulsed flow irrigation may facilitate the removal of the particles. The suction may be applied by a vacuum to evacuate the target area at a flow rate balanced with the flow rate of the introduction of the irrigation fluid.

The target area may be any location. In some embodiments, the target area may be anywhere within a urinary tract including, but not limited to, a kidney. In some embodiments, the target area may be a site in the body where particles are known or suspected to be located. In one example, the target area may be the site of a recent medical procedure that may result in particles (e.g., stone dusting) or an area where particles may be less likely to flow out naturally (e.g. the lower pole of the kidney). It should be noted that while kidney stones and stone fragments/dust are used for exemplary purposes, the present disclosure is not limited thereto. For example, the medical device and methods of use described in this disclosure may be used to remove any particles or other objects from any area of a body.

### Exemplary Embodiments

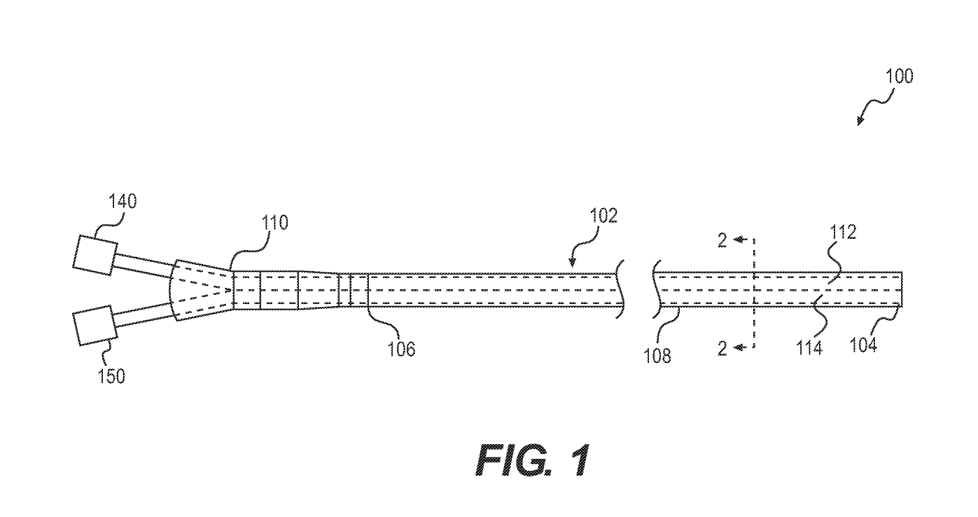

FIG. 1 illustrates an exemplary medical device 100 for removing stone dust. The device 100 may include a catheter 102. Catheter 102 may consist of a hollow, flexible, elongate tube 108 having independent first and second lumens 112 and 114. Tube 108 may have a distal end 104 and a proximal end 106 with dual lumens 112 and 114 therebetween. Proximal end 106 may be coupled to a handle portion 110, while distal end 104 remains open to allow flow of irrigation fluid and/or application of suction through lumens 112 and/or 114.

### A. The Handle Portion

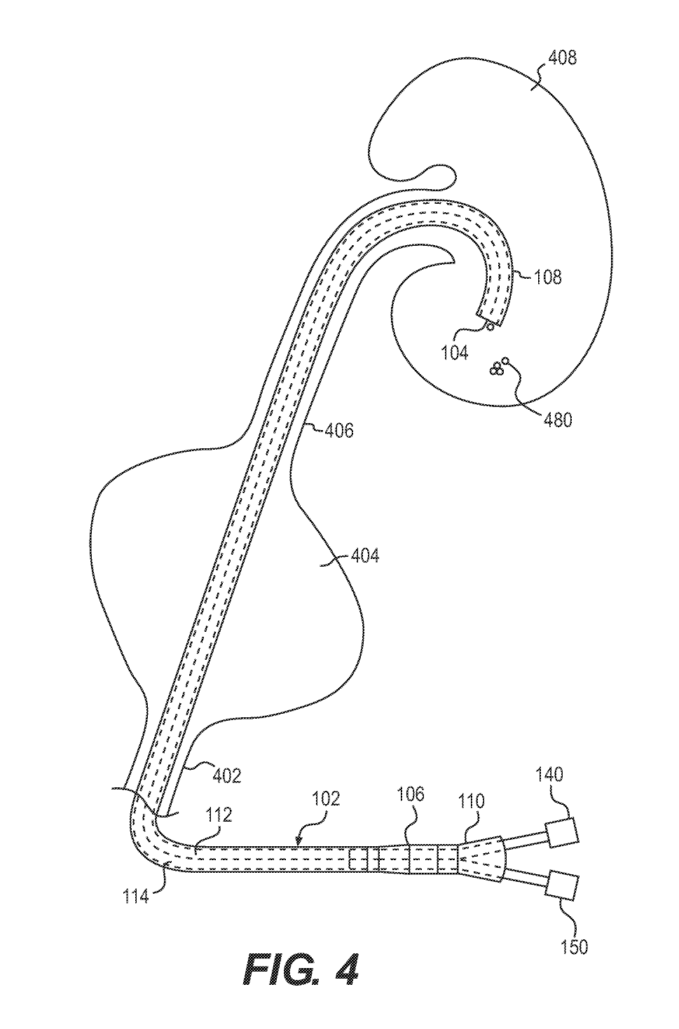

Handle portion 110 can be attached to tube 108 by, for example, welding, a locking configuration, use of an adhesive, or integrally forming with tube 108. The handle portion 110 may include two ports to place lumens 112 and 114 in fluid communication with a fluid supply assembly 140 and a vacuum source 150, respectively. The fluid supply assembly 140 and a vacuum source 150 will be described in further detail below with respect to FIG. 4. The handle portion 110 may include an actuating mechanism (not shown) to actuate one or more medical devices that may be located at the distal end of tube 108 or selectively expand tube 108, as desired. For example, the handle portion may include an actuating mechanism to power on or off an illumination device and/or imaging device.

### B. The Tube

Tube 108 may be circular, ovoidal, irregular, and/or any shape suitable to enter a body. Further, tube 108 may have a uniform shape along its length, or may having a varying shape, such as a taper at the distal end to allow to facilitate insertion within the body. Depending upon the particular implementation and intended use, the length of tube 108 may vary. The diameter of tube 108 may be tailored based on the body cavity. Similarly, depending upon the particular implementation and intended use, tube 108 can be rigid along its entire length, flexible along a portion of its length, or configured for flexure at only certain specified locations.

In one embodiment, tube 108 may be flexible, adapted for flexible steering within bodily lumens, as understood in the art. For example, tube 108 can be steered using a steering system (not shown) to move at least a portion (e.g., distal end 104) up/down and/or side-to-side. Additional degrees of freedom, provided for example via rotation, translational movement of tube 108, or additional articulation of bending sections, may also be implemented. Examples of such steering systems may include pulleys, control wires, gearing, and electrical actuators.

Tube 108 may be formed of any suitable material having sufficient flexibility to traverse body cavities and tracts. In general, tube 108 may be made of any suitable material that is compatible with living tissue or a living system. That is, the tube 108 may be non-toxic or non-injurious, and it should not cause immunological reaction or rejection. In some embodiments, tube 108 may be made of polymetric elastomers, rubber tubing, and/or medically approved polyvinylchloride tubing. Polymeric elastomers may be, for example, EVA (Ethylene vinyl acetate), silicone, polyurethane, and/or C-Flex.

Tube 108 may be designed to impose minimum risk to the surrounding tissues while in use. To this end, one or more portions of tube 108 may include atraumatic geometrical structures, such as rounded or beveled terminal ends or faces, to reduce trauma and irritation to surrounding tissues.

To effectively maneuver the tube 108 within a body cavity, the operator may need to know the exact location of the tube 108 in the body cavity at all times. To this end, one or more portions of the tube 108 may be radiopaque, such as by inclusion of barium sulfate in plastic material or inclusion of one or more metal portions, which provide sufficient radiopacity. Additionally or alternatively, distal end 104 of tube 108 may include radiopaque or sonoreflective markers (not shown). These markings facilitate detection of a position and/or orientation of the tube 108 within a patient's body, and an operator, with the aid of suitable imaging equipment, may track the path followed by tube 108. This may help the operator avoid potential damage to sensitive tissues. By using fluoroscopic guidance, the space within catheter 102 that would be needed for direct visualization (e.g., an imaging apparatus) may instead be used to maximize the size of the lumens and/or the flow rate of introduced fluid.

In some embodiments of the present disclosure, the distal end 104 of tube 108 includes visualization devices such as a camera and/or a light source. These devices may be integrally formed with the tube 108 or may attach to the distal end 104 using known coupling mechanisms. Alternatively, the visualization devices may be detachably introduced into tube 108 through lumen 112 and/or lumen 114 when required. For example, the proximal end of lumen 112 and/or lumen 114 may be forked to allow introduction of additional devices as well as a connection to either fluid supply apparatus 140 and/or vacuum source 150. Additionally or alternatively, lumen 112 and/or lumen 114 may include a side port(s) for introduction of additional devices.

Further, the tube 108 may include any suitable coating and/or covering. For example, the outer surface may include a layer of lubricous material to facilitate insertion through a body lumen or surgical insertion. Further, tube 108 may be coated with a biocompatible material such as Teflon. To inhibit bacterial growth in the body cavity, tube 108 may be coated with an antibacterial coating. Further, an anti-inflammatory substance may also be applied to the outer surface of the tube 108, if required.

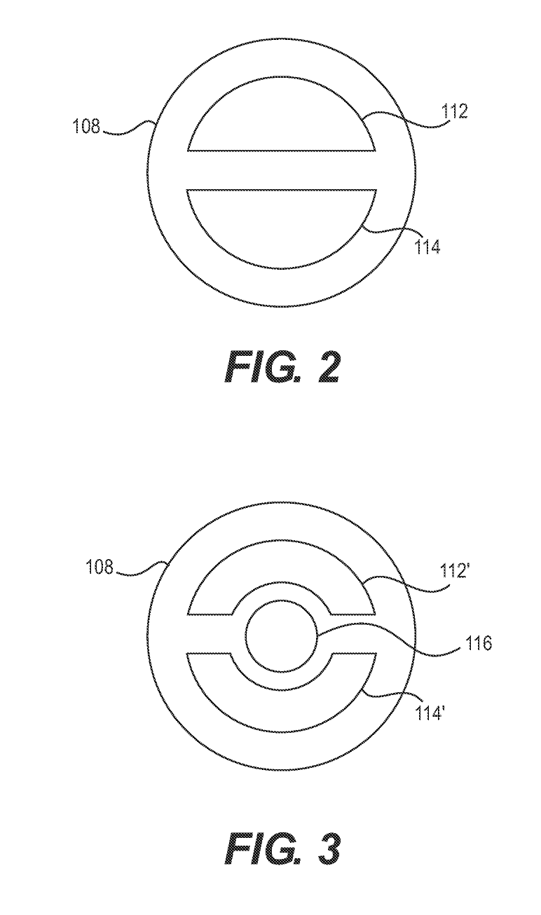

Lumens 112 and 114 may be defined by elongate hollow lumens that extend between proximal end 106 and distal end 104 of tube 108. While two lumens are illustrated in FIG. 1, tube 108 may include any number of lumens. The lumens included in tube 108 may be any size, shape, and/or in any configuration. Exemplary cross-sections of tube 108 and lumens 112 and 114 will be described in further detail below with respect to FIGS. 2 and 3.

FIG. 2 illustrates a cross-sectional view of tube 108 depicting dual-lumens 112 and 114. This view may, for example, be at line 2-2′ of FIG. 1, distal end 104, or proximal end 106. As shown, tube 108 may include two semi-circular shaped lumens 112 and 114. In the example shown in FIG. 2, lumens 112 and 114 have approximately the same cross-sectional shape and size. In some embodiments, lumen 112 may be have a different cross-sectional shape and/or size than lumen 114. Either lumen may be configured to supply fluid, apply suction, or both.

FIG. 3 illustrates an alternative cross-sectional view of tube 108 depicting lumens 112′, 114′, and 116. This view may, for example, be at line 2-2′ of FIG. 1, distal end 104, or proximal end 106. As shown, tube 108 may include a generally circular lumen 116 and two arc and/or curved shaped lumens 112′ and 114′. In the example shown in FIG. 3, lumens 112′ and 114′ have approximately the same cross-sectional shape and size. In some embodiments, lumen 112′ may be have a different cross-sectional shape and/or size than lumen 114′. It should be noted that lumens 112′, 114′, and 116 may have any shape or size and be in any configuration.

In the example illustrated in FIG. 3, lumens 112′ and 114′ may be configured to supply fluid, apply suction, or both. Lumen 116 may be configured to receive a medical device, tool, and/or instrument. In some embodiments, lumen 116 may receive a guidewire to facilitate location of the tube 108 in the body. As such, the guidewire may be inserted into the body before the insertion of tube 108 but after a medical procedure (e.g., Urolithiasis procedure, stone dusting, etc.), and the tube 108 may be tracked over the guidewire to position the tube 108. The guidewire may have been inserted into the patient for a related medical procedure and remain in the patient after the medical procedure was completed. It should be noted that any of lumens 112′, 114′, and 116 may be configured to supply fluid, apply suction, and/or receive/contain/enclose a guidewire.

Any lumen may include any suitable coating. For example, a lumen may include a layer of lubricous material to facilitate insertion of any instrument and/or device. In some embodiments, lumen 116 may be coated with a lubricous material to facilitate insertion of a guidewire.

### C. Insertion and Operation of the Medical Device

Referring to FIG. 4, an operator (e.g., a doctor or other medical personnel) may insert the distal end 104 of a catheter 102 into the patient's urethra 402. The operator may advance the catheter 102 so that the distal end 104 passes into and through the urinary bladder 404, into and through the ureter 406, and into the kidney 408. The operator may position the distal end 104 of the catheter 102 within the patient's kidney 408. The operator may position a distal opening of lumen 112 and/or a distal opening of lumen 114 proximate a target area. A target area may include, but is not limited to, the site of a recent Urolithiasis procedure, a site where stones and/or stone fragments 480 are known or suspected to be located, and/or the lower pole of the patient's kidney 408.

It should be noted that, though FIG. 4 illustrates distal end 104 of tube 108 within a kidney 408, the present disclosure should not be limited thereto. For example, the introduction of fluid and/or application of suction, described below and throughout this disclosure, may be performed anywhere within a patient.

The operator may connect lumen 112 to the fluid supply assembly 140. The fluid supply assembly 140 provides fluid, through lumen 112, to the distal end 104 of tube 108 and into kidney 408. The fluid supply assembly 140 may be any device and/or devices that can supply fluid to lumen 112. The fluid supply assembly 140 may include, but is not limited to, a fluid source, a pump, a control system, a heat exchanger, a filter, a temperature sensor, a pressure sensor, a supply line, and/or various user input devices. In some embodiments, the fluid supplied is a saline solution, for example, 0.9% saline.

The fluid may be provided to the supply lumen 112 in a continuous, constant flow, or as a pulsed flow. In some embodiments, both a continuous and pulsed flow may be used. For example, an operator may initially introduce the fluid in a continuous flow, but transition to a pulsed flow later in the procedure (or vice versa: starting with pulsed and switching to continuous). In some embodiments, there may be a plurality of fluid introduction lumens, allowing for the simultaneous introduction of fluid as continuous flow and as pulsed flow.

A pulsed flow may dislodge stone fragments/dust adhering to various surfaces within the patient's body. The pulsed flow may create turbulence. The turbulence to stir up any stone fragments/dust which may be located in an area where the stone fragments/dust may be less likely to flow out naturally (e.g. the lower pole of the kidney). The turbulence may aid in keeping stone fragments/dust 480 in suspension so that they may be more effectively suctioned out through lumen 114.

The fluid may be provided to the supply lumen 112 at a variety of flow rates. In some embodiments, the flow rate may be pulsed at a regular interval, e.g., every few minutes. The pulsed flow may be a flow that is either intermittently interrupted or simply reduced in rate on an intermittent basis. The flow rate may be pulsed at complex flow rate patterns such as periodic and aperiodic oscillatory patterns. For example, a pulse interval (time between pulse cycles) may be adjustable and may range e.g. from 100 pulses per second to 1 pulse cycle every 2 seconds. A pulse pattern may be pre-set, determined in real-time by the operator, or may be actively controlled and optimized based on parameters collected from sensing mechanisms and implemented by a processor.

A pulsed flow may be created in any way. In one embodiment, the pulse flow may be created by a mechanical pump. The mechanical pump may apply and release pressure on the fluid at intervals. The mechanical pump may be operated and controlled manually or a processor may control the pattern, duration, intensity, intervals, etc. of the pulses.

Referring again to FIG. 4, the operator may connect lumen 114 to a vacuum source 150 (e.g., house vacuum, vacuum pump, etc.). The vacuum source 150 may provide suction and allow the operator to vary the suction. The vacuum source 150 may be located near the patient or may be located remotely (such as a vacuum source located on a wall). In some embodiments, lumen 114 and vacuum source 150 may be connected via a conduit. The conduit may be made of a flexible material (for example, a polymeric tube), a rigid material, or a combination of both flexible and rigid materials. In some embodiments, the conduit may be braided or wound with plastic or metal fibers to improve conduit's resistance against kink-formation or against collapse under vacuum pressure. In some embodiments, the conduit may include coatings on its inside or outside surface for various purposes, for example, for protection against corrosion and/or by body fluids. In general, the conduit may have any dimension suitable for its intended use. In some embodiments, an elongated polymeric or polypropylene tube may serve as the conduit.

The flow rate of the introduced fluid may be balanced with the flow rate caused by the vacuum. Balanced flow rates can be substantially equal, but need not be exactly equal. As used herein, the terms “about,” “substantially,” and “approximately,” may indicate a range of values within +/−5% of a stated value. A balanced flow rate may be any flow rate that prevents harm to the patient. For example, a balanced flow rate may be any flow rate of the introduction of fluid in relationship to the flow rate of the suction that prevents hydronephrosis and/or prevents the kidney from collapsing due to no fluid in the system, as known in the art. The balanced flow rate may assist in maintaining a pressure equilibrium during operation of the device. In some embodiments, a pressure sensor may also be located at or near the target area and/or distal end 104 to assist in maintaining a pressure equilibrium.

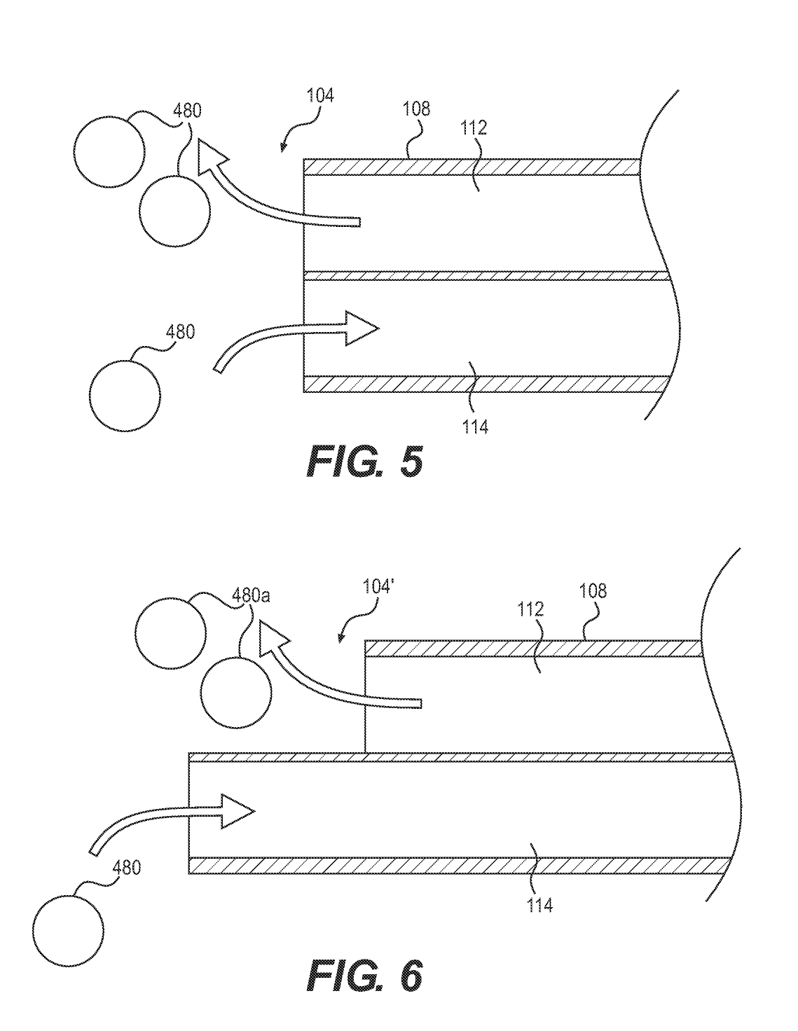

Referring to FIGS. 5 and 6, after tube 108 is advanced through the body, into the kidney 408 and is proximate to stone fragments/dust 480, the operator may turn on the fluid supply assembly 140 to introduce fluid through lumen 112 to the target area and/or the operator may turn on the suction to pull the stone fragments/dust 480 into lumen 114.

In FIGS. 5 and 6, lumens 112 and 114 each have one distal facing opening. It should be noted however that lumens 112 and 114 may have any number of openings within the human body in any configuration. For example, lumens 112 and/or 114 may have side ports for the introduction and/or suction of fluids and objects. It should also be noted that openings may be substantially perpendicular to the tube (e.g., as shown in FIG. 5), may be tapered or angled, or may be in any other suitable orientation. Further, lumens 112 and 114 may be any size or shape. For example, a vacuum lumen with a larger cross-sectional area may prevent stone fragments/dust from obstructing the flow. It should be noted that in such a configuration, balanced flows may still need to be maintained despite the differing sizes of lumens 112 and 114.

As shown in FIG. 5, in some embodiments, lumens 112 and 114 may terminate at approximately the same position on the distal end 104 of tube 108. As shown in FIG. 6, in some embodiments, the distal end 104′ may include lumens 112 and 114 that are be staggered. This staggered configuration may provide improved suction of stone fragments/dust 480. For example, a staggered configuration may provide the desired effect of maintaining a pressure equilibrium through introduction of fluid, but avoid the undesired effect of fluid introduction pushing stone fragments/dust away from the opening of vacuum lumen 114. Additionally or alternatively, a staggered configuration may push stone fragments/dust that are located proximal to the opening of vacuum lumen 114 (e.g., stone fragments/dust 480a of FIG. 6) to a position distal to the opening of vacuum lumen 114, thus allowing stone fragments/dust 480a to be suctioned out of the body into lumen 114.

After suctioning stone fragments/dust 480, the operator may reposition the distal end 104 of tube 108. The purpose of repositioning the distal end 104 may be to reach stone fragments/dust that the device was previously unable to suction out of the body and into lumen 114. For example, some stone fragments/dust may be positioned proximally to the distal opening or positioned too distally to be captured by applied suction. After the operator repositions the distal end 104 of tube 108, the process of introducing fluids and applying suction may begin again, at any flow rate or pulse type suitable. A operator may reposition the distal end 104 of tube 108 any number of times.

### D. Exemplary Method of Operation

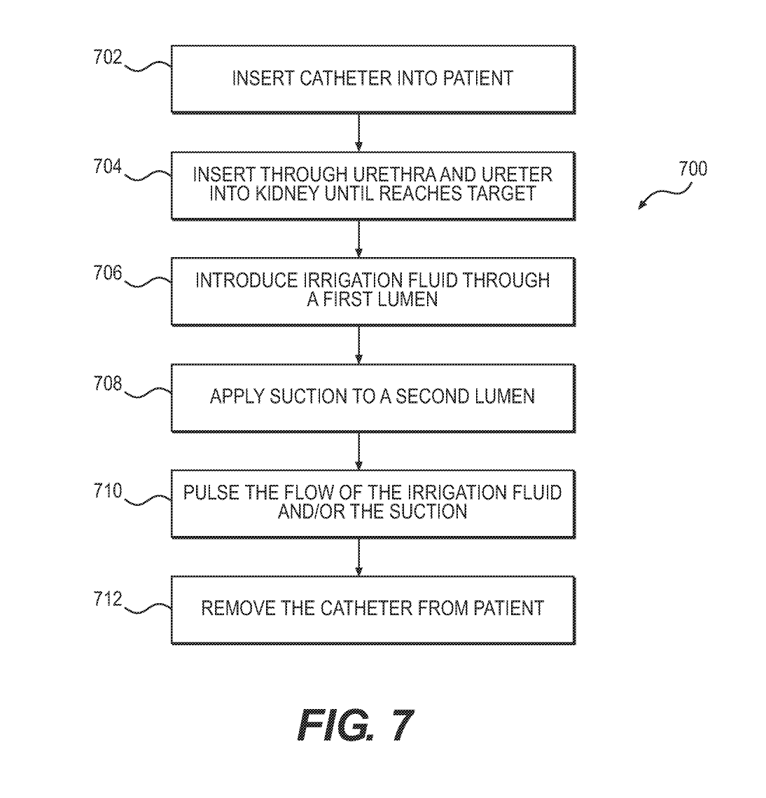

FIG. 7 illustrates an exemplary method of use of a medical device for removal of stone fragments/dust. For purposes of discussion, method 700 will be described using medical device 100 of FIG. 1 (including lumens 112 and 114), as described above, but method 700 is not intended to be limited thereto. As shown in FIG. 7, method 700 includes steps 702, 704, 706, 708, 710, and 712. However, it should be noted that method 700 may include more or fewer steps as desired for a particular implementation and the order of the steps may be varied.

Method 700 may commence after completion of a medical procedure that created stone fragments/dust (e.g., Urolithiasis procedure, stone dusting, etc.).

In some embodiments, the medical device that created the stone fragments is completely removed, prior to beginning step 702 of method 700. In other embodiments, a guidewire remains within the body. In other embodiments, the medical device that performed this procedure was introduced through a lumen in the catheter 102 (e.g., lumen 116).

In step 702, at least a portion of tube 108 of catheter 102 may be inserted into a patient's body. In step 704, the distal end 104 of tube 108 may be further inserted through the urethra and ureter, and into the kidney until it reaches the target region. In some embodiments, the distal end 104 of tube 108 may reach the target area by moving over a guidewire. The target region may be, for example, a site of a recent medical procedure (e.g., Urolithiasis procedure, stone dusting, etc.) or an area where stone fragments/dust may be less likely to flow out naturally (e.g. the lower pole of the kidney). Once the device is at the target region, the operator may introduce fluid (e.g., irrigation fluid) through a first lumen of tube 108 (step 706). At step 706, the fluid may be introduced in a continuous flow. In some embodiments, the fluid may be initially introduced in a pulsed flow. In step 708, suction may be applied to a second lumen of tube 108 (e.g. lumen 114). The suction may be applied at a flow rate balanced with the flow rate of the introduced fluid. It should be noted that steps 706 and 708 may be performed in any order. For example, step 708 may proceed step 706 or steps 706 and 708 may be performed concurrently. As steps 706 and 708 are performed, fluid as well as stone fragments/dust (natural occurring or a result of the medical procedure) may be suctioned into the second lumen and the fluid introduced from the first lumen may maintain a pressure equilibrium.

In step 710, the flow of the introduced irrigation fluid and/or the application of suction may be pulsed. The pulsed flow may allow the stone fragments/dust to be suctioned out more effectively. For example, the pulsed flow may keep the stone fragments/dust in suspension. It should be noted that step 710 may be eliminated from this method and the flow may remain constant and continuous throughout the procedure.

At any point, an operator may additionally choose to move the device within the patient. For example, an operator may choose to move the distal end 104 lower into the kidney or to location in which additional stone fragments/dust have accumulated. Any or all of steps 706-710 may be repeated at the new location. Once an operator determines no more stone fragments/dust can and/or should be removed, method 700 may proceed to step 712 and the catheter may be removed from the patient's body.

The many features of the disclosure are apparent from the detailed specification, and thus, it is intended by the appended claims to cover all such features of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of removing stone dust from a patient's body, comprising:
positioning a medical device at a target area within the urinary tract, the medical device including a first lumen and a second lumen;
introducing fluid through the first lumen; and
applying suction to the second lumen.

2. The method of claim 1, wherein a flow rate through the first lumen is balanced with the flow rate through the second lumen.

3. The method of claim 1, further comprising:
pulsing a flow of the fluid through the first lumen.

4. The method of claim 3, wherein the introduction of fluid through the first lumen includes pumping the fluid from a fluid source.

5. The method of claim 3, wherein the flow of the fluid is pulsed in an aperiodic oscillatory pattern.

6. The method of claim 3, wherein the flow of the fluid is pulsed in a periodic pattern.

7. The method of claim 1, wherein a distal opening of the first lumen is at the same axial position as a distal opening of the second lumen.

8. The method of claim 1, wherein a distal opening of the first lumen is positioned proximally to a distal opening of the second lumen.

9. A method of using a medical device to remove a particle from a subject's body, the medical device including a first lumen and a second lumen, comprising:
positioning the medical device proximal to a target area;
pulsing fluid through the first lumen; and
applying suction to the second lumen.

10. The method of claim 9, wherein the target area is within a kidney.

11. The method of claim 9, wherein the target area is the lower pole of the kidney.

12. The method of claim 9, wherein the fluid is pulsed in an aperiodic oscillatory pattern.

13. The method of claim 9, wherein the fluid is pulsed in a periodic pattern.

14. The method of claim 9, wherein a distal opening of the first lumen is at the same axial position as the distal opening of the second lumen.

15. The method of claim 9, wherein a distal opening of the first lumen is positioned proximally to the distal opening of the second lumen.

16. A method for removing fragments of a kidney stone, comprising:
after the completion of a medical procedure that results in small fragments of at least one kidney stone, positioning a medical device at a target area within the urinary tract;
introducing fluid; and
applying suction.

17. The method of claim 16, wherein the target area is the location of the completed medical procedure.

18. The method of claim 16, further comprising a lumen configured to receive at least one of an instrument, a medical device, and a guidewire.

19. The method of claim 18, wherein positioning the medical device, further comprises:
guiding the medical device over a guidewire.

20. The method of claim 16, wherein the medical device includes radiopaque or sonoreflective markers.
